(19) United States
(12) Patent Application Publication
(10) Pub. No.: US 2018/0161796 A1
(43) Pub. Date: Jun. 14, 2018

(54) MICRO-NOZZLE ASSEMBLY WITH FILTER

(71) Applicant: The Technology Partnership Plc, Royston, Hertfordshire (GB)

(72) Inventors: Daniel STRANGE, Royston, Hertfordshire (GB); Robert SELBY, Royston, Hertfordshire (GB); Romain GUION, Royston, Hertfordshire (GB); William RICHARDSON, Royston, Hertfordshire (GB); Lawrence BAKER, Royston, Hertfordshire (GB)

(21) Appl. No.: 15/736,623

(22) PCT Filed: Jun. 15, 2016
(86) PCT No.: PCT/GB2016/051778

§ 371 Date: Dec. 14, 2017

(30) Foreign Application Priority Data
Jun. 15, 2015 (GB) 1510445.8
Jun. 15, 2015 (GB) 1510448.2

(51) Int. Cl. B05B 15/40; A61M 11/00; A61M 39/24; A61F 9/00

(52) U.S. Cl. CPC B05B 15/40; A61M 11/006; A61M 39/24; A61M 2205/3334; A61M 2205/7545; A61M 2207/00; A61F 9/0008

(57) ABSTRACT

A nozzle assembly, configured to receive fluid at pressures of greater than 1 bar. The nozzle assembly comprises a first perforate element (1) comprising one or more orifices (2), each orifice having an inlet and an outlet and a diameter of no more than 100 μm, and at least one second perforate element (4) further comprising a plurality of orifices of a smaller size than the one or more orifices of the first perforate element (1) and having a larger number of orifices than the first perforate element (1). The second perforate element (4) is configured to act as a filter and the second perforate element (4) is attached to the first perforate element (1). A perpendicular distance between the first and second perforate elements (4) is less than the diameter of the largest orifice of the first perforate element (1).

### FIELD OF THE INVENTION

The present invention relates generally to aerosol systems and, more particularly, to aerosol systems comprising a nozzle assembly for producing an aerosol, a filter for preventing any particles from blocking the nozzle and/or a valve for producing a check valve and a flow regulation function.

### BACKGROUND

Aerosol systems that can generate a slow moving mist, such as those described in PCT/GB2015/051413 and GB1420266.7, are highly effective and user-friendly methods of delivering pharmaceutical ingredients to the lungs, nose, eyes, and skin or mouth. The mist is monodisperse and is much more controllable than a mist produced by a typical pump spray. As such, delivery can be targeted with fast uptake and undesirable effects, such as over spray (in the case of nasal delivery) or poorly controlled droplet distribution at the start up and end of the droplet delivery, can be avoided.

Such slow moving mist aerosol systems operate by forcing liquid at high pressures through micro-structured nozzles comprising orifices with diameters of 100 μm or less. The pressure is often generated by use of a compression spring acting on a piston to generate a substantially constant pressure. Such a spring can be primed by the user of the device.

One of the key challenges in the design and manufacturing of slow moving mist aerosol devices is preventing the micro-structured nozzles from becoming blocked when liquid is forced through them under high pressure. Contamination can come from many sources, such as wear, manufacturing debris, and formulation precipitation. Contamination can cause impaired performance and safety issues. Ordinary room air typically contains over 35,000 particles per square metre with a diameter of 10 μm or greater and each of these particles could potentially block a nozzle of a similar size. A statistical filter, such as depth filters having solely a nominal rating, may prevent most particles from going through, but a single particle going past the filter can block the nozzle and jeopardize the proper operation of the entire device in which the filter is used.

Filtering is critical in many applications where contamination needs to be removed from a fluid flow. Contamination can come from many sources, such as wear, manufacturing debris, and formulation precipitation. Contamination can cause impaired performance and safety issues. For example in the presence of small nozzle downstream (e.g. <100 μm), any particles of a size above a small fraction of the nozzle size can potentially block it. A statistical filter, such as depth filters having solely a nominal rating, may prevent most particles from going through, but a single particle going past the filter can block the nozzle and jeopardize the proper operation of the entire device in which the filter is used.

Also, some formulations, such as intranasal steroids, comprise suspensions of particles. Although the particles in the suspensions are generally smaller than the nozzle, they can agglomerate together to form larger aggregate particles that can to block a nozzle.

Hence, it is necessary to provide a filter in slow moving mist aerosol spray systems to prevent any particles of a similar size to the nozzle orifice blocking the nozzle under high pressure. U.S. Pat. No. 7,316,067 describes a method of forming a perforate membrane, which can be used as a filter, wherein the holes formed by laser drilling.

In conventional spray pumps gaskets or seals often lie between the nozzle and filter and may themselves generate wear particles which can block the system at a later point in time. Further, when a constant force is applied to a typical spray pump, the force compresses any compliant seals and any trapped air bubbles, at the same time as forcing liquid through the nozzle. When the force is removed, the compressed seals and air continue to apply a pressure to the liquid that gradually decays with time. Liquid can continue to be forced through the nozzle for several seconds afterward, leading to undesirable continued droplet distribution.

The combined nozzle and filter system should be low-cost, ideally less than 0.10 USD, in volumes of less than 5 million units per year in order to be suitable for use in the consumer healthcare market. Both U.S. Pat. No. 7,896,264 and US publication number US20050178862 describe methods of forming micro-structured high pressure nozzles with built-in filter functions. However, these nozzle assemblies are formed using micro-machining techniques and techniques using anodic bonding of glass wafers. Both techniques require substantial capital expenditure (Cap-Ex) investments and each nozzle likely costs substantially more than 0.10 USD at low volumes.

A low cost method of forming a high performance nozzle assembly with a built in filter function and consistent spray properties is needed.

In addition to filters, valves are useful for a variety of applications in aerosol systems, for instance one-way valves enhance the performance of pumping or help avoid air ingress through a nozzle of an aerosol generating device when filling a metering chamber of the device. Although small holes at a liquid-air interface at the nozzle provide some ‘valving’ effect due to the capillary pressure, it is limited to a fraction of a bar for holes as small as 10 μm, and it acts only when the holes of the nozzle are between two phases. For example, a hole with liquid on one side and gas on the other side will have some valve effect, but a hole immersed in a liquid or a fluid will not.

A practical example can be found both with the priming steps and the normal actuation of aerosol technologies such as those of the device described in international application number PCT/GB2015/051413. A typical metered pump spray requires two valves for normal operation, a first valve near the nozzle and second valve on a piston. When the metering chamber is filled the first valve must be closed so that a partial vacuum is created in the metering chamber which pulls fluid in through the second valve from the dose reservoir. When fluid is ejected from the metering chamber the second valve closes generating high pressure and the first valve must open to allow fluid out of the chamber. The typical metering volume of a device such as that described in PCT/GB2015/051413 can be as small as 10 μL, which is substantially smaller than the dead volume of a typical off the shelf ball valve. The use of a typical off the shelf valves would create a significant increase in dead volume, which typically results in more priming shots being required to fill the metering chamber, and in inconsistent performance through life resulting from the additional space for bubbles to become trapped.

A need for a different type of valve in aerosol systems arises where flow regulation is required. For example, there may be a need to increase flow resistance as flow rate increases and decrease flow resistance as flow rate decreases so as to maintain a consistent flow rate. The need for a controlled flow rate extends beyond aerosol systems to any application where a stabilised flow rate is needed. Examples of industrial applications are, e.g. when the flow rate of a fluid feed must not exceed a certain threshold, as could be the case in heat exchanges, combustion and chemical reactions. Examples of medical applications include controlling the inhalation flow rate or controlling the injection of fluids into the body.

The combined need to use a valve and a filter is very common, but it conventionally requires several parts assembled together and, therefore, a more complex and expensive manufacturing process. A typical example is described in international application number WO2014107436A1, where an upstream filter is provided for preventing contamination from affecting the sealing of a check valve membrane. Another example can be found in the food industry in which pressure relief valves coupled with filters are used on products packaged in airtight packaging that normally tend to give off gas, such as coffee. US application number US2009169693A1 describes such a valve with a filter.

Dead volume is critical to many applications, particularly in those applications that use a valve. An example is with devices that have a priming step, as a large dead volume increases the flushing time and volume of flushing fluid wasted. Another example is for pressurised environment, since any compliance will affect pressure, possibly randomly if compliance is not consistent. Reducing the dead volume enables reduction of the compliance, for instance by reducing the pressurised area in the case of a compliant chamber, or by reducing the probability of trapping air and by minimising the maximum volume of trapped air bubbles. For example in the context of a 5 μl metering chamber swept by a piston, a bubble as small as 1 μl will cause at most 80% of the fluid to be delivered at the target pressure, while the remaining 20% of the fluid will be released at pressures several orders of magnitude below the target. This could lead to a variety of issues in practice, in particular, in the context of drug delivery or in microfluidics, for example an uncontrolled dose variation or a variation in the droplet size distribution for an aerosol generating device.

Known valves, filters and nozzles are often not easily tuneable. Low volume applications such as a niche product or a personalised product may require bespoke specifications that would yield high costs if modifications of the manufacturing or assembly lines were required. In particular, the possibility to tune the cracking pressure of a one-way valve is often key, e.g. to avoid early firing or to control the minimum pressure downstream of the valve. The resistance of a valve, its leakage rate and the range of operating pressures in both directions are four other important parameters. Being able to control those with little setup required would be extremely valuable. As for the filters and nozzles, they often need to be tuned in terms of hole(s)/pores sizes, and number of holes/pores, e.g. to control the diameter of a jet or flow resistance.

US application number US2008011667A1 is one of many examples in which filters and valves are described which assembled one after the other. Such assemblies require many parts, and always involve an outer member to hold and support the filter member and valve together. This results in increased cost, bulkiness, and dead volume. More specifically they can involve membrane valves such as those described in international application number WO2014107436A1 or European application number EP2679273A, in which a membrane is clamped within a cavity and a filter is added in the fluid feed upstream of the check valve. Again, such assemblies are complex and can be expensive to manufacture.

Membrane (or diaphragm) valves are sometimes perforated with holes, such as the valve described in U.S. Pat. No. 4,141,379A, and a membrane check valve clamped between two parts of the housing is used and seals against the housing, which acts as a valve seat. The necessity to use the housing to clamp and/or seal the membrane valve within the housing is an integral part of the valve function and, because of this, the valve is not a drop-in part. In addition the valve seat does not provide filtering, nor does the perforated membrane. As such, there is provided a microporous screen, downstream of the valve, that acts as a filter.

Accordingly, there is a need to provide an improved valve and a filter system which overcomes one or more of the above outlined issues with known valve and filter systems. There is a need for a low cost (<0.05 USD) and compact (e.g. containable in a millimetric envelope) valve in series with a filter and/or a nozzle (such as a sub-micrometric nozzle), with a minimised assembly dead volume.

### SUMMARY OF THE INVENTION

According to an aspect of the invention, there is provided a nozzle assembly, configured o receive fluid at pressures of greater than 1 bar, the nozzle assembly comprising: a first perforate element comprising one or more orifices, each orifice having an inlet and an outlet and a diameter of no more than 100 μm; and at least one second perforate element further comprising a plurality of orifices of a smaller size than the one or more orifices of the first perforate element and having a larger number of orifices than the first perforate element; wherein the second perforate element is configured to act as a filter, wherein the second perforate element is attached to the first perforate element, wherein a perpendicular distance between the first and second perforate elements is less than the diameter of the largest orifice of the first perforate element.

The invention is low cost, containing only 2 elements, and is easy to assemble and manufacture at high throughput—multiple nozzle assemblies can be assembled and attached together before being punched out for further handling. Furthermore, the invention is a self-contained unit. The nozzle assembly must be assembled in clean conditions in order to minimise the risk of particle ingress into the space between the nozzle and filter (which could then block the nozzle in use), but once assembled it can be handled with little further risk of ingress, particularly as the filter is an absolute filter. The low cost of the unit means it can be tested together as one and scrapped if it fails an in process check, with little effect on the overall device cost.

Finally, the invention also has advantageous performance characteristics in use. The assembly is mechanically robust and can withstand pressures of up to 300 bar. The viscous losses through the pressure assembly are typically small compared to the pressure required to aerosolise fluid out of the nozzle (<20%). The dead volume between the nozzle and filter is small, which reduces the amount of priming shots that must be delivered, and also provides little space for bubbles to collect and interfere with the spray generation. The lack of moving parts eliminates any mechanisms that may generate wear particles. The nozzle assembly can be made solely from stainless steels so it is corrosion resistant. Lastly, the filter can be placed directly in contact, or very close to the nozzle preventing the ingress of bacteria into the device and allowing for preservative free delivery.

According to a further aspect of the invention, there is provided a nozzle assembly through which fluid is forced at pressures greater than 1 bar, the nozzle assembly comprising: a first perforate element comprising one or more orifices, each orifice having an inlet and an outlet and a diameter of no more than 100 μm; at least one second perforate element further comprising a plurality of orifices of a smaller size than the one or more orifices of the first perforate element, the second perforate element having a larger number of orifices than the first perforate element, the second perforate element being arranged to act as a filter; and at least one intermediate element that separates the first and second perforate elements, wherein the second perforate element is attached to either the intermediate element or the first perforate element, wherein a perpendicular distance between one or more of any two consecutive perforate and/or intermediate elements is less than the diameter of the largest orifice of the first perforate element.

The addition of the intermediate element provides further design flexibility over the nozzle assembly. The distance between the nozzle and filter can be tightly controlled so that the two remain separated under high pressure even with small amounts of elastic deformation, or conversely so that the two move together under high pressure inducing flow restriction. It also facilitates the use of alternate materials and manufacturing methods that can be used to seal between the nozzle and filter. The nozzle and filter could be combined together with an intermediate element made from a thermoplastic elastomer (TPE) using a continuous process such as reel to reel over moulding.

The present invention describes a low-cost nozzle assembly, wherein a nozzle and filter are attached together, sometimes via one or more intermediate elements.

Although an intermediate element, such as a washer, is not an essential feature, in a preferred embodiment of the nozzle assembly, a thin (20 μm-500 μm in thickness, preferably 100 μm in thickness) first perforate element is affixed to a thin (10 μm-300 μm thick, preferably 20 μm thick) intermediate element, such as a washer. The first perforate element comprises one or more orifices with a diameter in the range of 1 μm-100 μm, and preferably 10 μm.

The intermediate element is then affixed to a second perforate element comprising a larger number of orifices that are smaller in diameter than the orifices of the first element, preferably 5 μm in diameter, for example where the orifice of the first element with the smallest diameter has a diameter of greater than 5 μm. The second perforate element is disposed on a liquid side of the assembly, namely the side of the assembly configured to abut with a metering chamber defined by a tube, and acts as an absolute filter, preventing particles entering the nozzle assembly from the metering chamber and blocking the orifice or orifices of the first perforate element.

In an alternative embodiment of a nozzle assembly, the first perforate element comprises a moulded part affixed to a second perforate element. The moulded part may be made of plastic. The moulded part comprises at least two holes with hydraulic diameters in the range of 5 μm to 100 m, and preferably 30 μm in diameter. The projected areas of the holes at least partially intersect at the outlet side of the nozzle and filter assembly, such that liquid forced through each hole will form jets which impinge on each other, creating a slow-moving soft mist. The second perforate element comprises a much larger number of orifices that are smaller in diameter than the two orifices in the moulded part. The second perforate element acts as an absolute filter and prevents particles entering the orifices of the moulded part and blocking them.

In all embodiments, the various parts and elements are preferably affixed together by laser welding, although bonding and clamping are also possible, providing a sealed unit that can be easily handled after assembly. It should be noted that, although a sealed unit is desirable, spot welds can also be used as long as the remaining gaps between the elements are smaller than that of the nozzle orifice.

Furthermore, the orifices can be fabricated in each of the perforate elements using laser drilling. This operation could potentially be performed using the same laser as that used for laser welding, where the elements are laser welded together. Welding is preferable to other bonding processes, such as glue, as it is a clean process with little chance of particles/material generated during the sealing process blocking the nozzle. Moreover, welding avoids the need to test the materials (such as glue or any other bonding material which may be used) for extractables and leachables that may contaminate any liquid formulation, during use of the nozzle assembly. Welding is also preferable to clamping as the nozzle filter assembly can be handled as a standalone unit, without risk of particulates entering this space on the factory floor, whereas clamping requires a separate clamp means for clamping the perforate elements together. Furthermore, very strong joins can be formed by welding between the nozzle and filter which can resist high pressures. Laser welding is particularly advantageous as it has a very fast cycle time (sub one second) and can be performed as a masked process, using a suitable mask, facilitating high volume production.

Although laser welding is preferred, ultrasonic, electron beam and thermal welding also present viable alternatives.

The present invention is low-cost: the bill of materials is very low as the various elements can be stamped from large sheets of metal or polymer and the capital investment required for manufacturing is also small as it only requires a suitable laser system for both welding and drilling processes. It is easy to manufacture and assemble in high volumes. A nozzle assembly in accordance with the present invention has little dead volume in the space between the nozzle and the filter, and is substantially rigid such that it will not expand substantially under high pressure, storing fluid that would otherwise exit through the nozzle. It also presents little additional flow resistance, compared to that of a conventional nozzle assembly, as the flow path through the nozzle assembly is relatively short, and the combined area of all of the orifices of the filter of the nozzle assembly is substantially greater than that of the one or more orifices of the nozzle. The invention is easier to assemble than conventional methods of using a gasket or o-ring, and it can be transported around an assembly line without risk of particulates entering the space between nozzle and filter.

According to a further aspect of the invention, there is provided a valving and filtering device comprising a first perforate member; and a second perforate member attached to the first perforate member, wherein the first perforate member is more flexible than the second and, wherein orifices of at least one of the first and second perforate members have a hydraulic diameter between 0.1 μm and 1 mm per hole.

According to a further aspect of the invention, there is provided a valving device comprising a first perforate member; and a second perforate member attached to the first perforate member, wherein the first perforate member is more flexible than the second and, wherein the first perforate member is configured to be pushed onto the second perforate member as flow rate increases, thereby increasingly restricting the flow.

According to a further aspect of the invention, there is provided valving device comprising: a first perforate member; a second perforate member attached to the first member; and a third perforate member, wherein one or more of the first perforate member and the second perforate member are attached to the third perforate member, wherein the first perforate member is more flexible than the second perforate member and the third perforate member and, wherein the first perforate member is disposed between the second and third perforate members.

### BRIEF DESCRIPTION OF DRAWINGS

Embodiments will now be described, by way of example only, with reference to the accompanying drawings, in which:

FIG. 1 shows a nozzle assembly;

FIG. 2 shows an alternative nozzle assembly;

FIG. 3 shows a further alternative nozzle assembly;

FIG. 4 shows a preferred embodiment of a valving and filtering device;

FIG. 5 shows alternative embodiments of the valving and filtering device;

FIG. 6 shows a further alternative embodiment of the valving and filtering device;

FIG. 7 shows further alternative embodiments of the valving and filtering device; and

FIG. 8 shows flow rate verses pressure graphs illustrating the operation of embodiments of the valving and filtering device.

### DETAILED DESCRIPTION

The words “perforate” and “perforated” encompass any form of “aperture(s)”, “orifice(s)”. or “perforation(s)”, which can be any shape, e.g. holes, slots/slits, meshes, or any gap/opening. ‘Aperture’, ‘perforation’, ‘orifice’ and ‘hole’ are used interchangeably, in the singular or plural form, to refer to either a single or multiple apertures/perforations/orifices/holes.

For filtering or for the nozzle(s), having small and controlled apertures is often crucial. Holes of a few hundred microns can be moulded or machined. Electroforming is also a solution and enables to reach smaller holes. For holes going down to the micron size, laser drilling can be used, with or without electro-polishing. Other techniques, such as track etching and sintering, enable the production of holes below the micron size.

The word “attached” is intended to encompass any method that enables two members or parts to be maintained/bonded together. This includes clamping, adhesive/solvent bonding (e.g. using glue), welding (e.g. ultrasonic, hot air welding), co-moulding, soldering, brazing, bonds of any kind (diffusion, isothermal solidification, transient liquid phase, exothermic, etc.), cladding, heat sealing, induction sealing, and any other suitable known means as would be understood by the skilled person.

The seal between the two members does not need to be perfect. The valving function can be performed if the aperture area of the leak path (i.e. the imperfect seal) is smaller than the aperture area of the second member. The filtering function can be achieved if the individual leak paths are smaller than the filter size.

The expression “flexing member” is used to designate the member deflecting more than other members (when realising the desired function) as a result of a given pressure drop across the system (or a given flow rate). Indeed, there are cases in which both members flex, particularly in high pressure environments. More details on various ways to control the flexing are provided herein. It is important to note that ‘flexing’ includes, but is not limited to, the “flexibility” of each member, as there are embodiments of the valving and filtering device that will generate different pressures drops on each of the two members of the system, e.g. with different aperture areas on each member.

FIG. 1 shows a nozzle assembly. The nozzle assembly comprises an assembly of three elements, preferably in the form of discs, which are welded together. Alternatively the discs may be bonded, soldered or brazed together; co-moulded together; or even clamped together by an external element. Each disc is approximately 4 mm in diameter.

The first disc or element 1 is configured to act as a nozzle. It comprises of a thin plate of 20-300 μm in thickness, typically 100 μm thick, with a single central orifice 2 that has a hydraulic diameter of 2-100 μm. The orifice 2 may be formed through a laser drilling process, or alternatively via electroforming or micromoulding, EDM, micro machining, etching.

The second disc or element 4 is configured to act as an absolute filter. It comprises a thin plate of 10-300 μm in thickness, typically 50 μm, which comprises a plurality of orifices 5, typically a thousand, that are smaller in diameter than those of the first disc 1 (preferably half the diameter of the orifices of the first disc, i.e. 5 μm in diameter if the diameter of the orifices of the first disc is 10 μm). The orifices 5 of the second disc 4 prevent larger particles from entering the cavity between the second disc 4 (filter) and first disc 1 (nozzle) where they might cause blocking of the first disc (nozzle orifice) 1.

The orifices 5 of the second disc 4 can be formed in the second disc 4 through a laser drilling process, though other processes such as electroforming, micro-moulding and track etching are also suitable.

A third disc or element 3 is an intermediate disc or element which acts to separate the first disc 1 and the second disc 4, while still permitting fluid communication between the first disc 1 and the second disc 4. The third disc 3 comprises a thin washer of 10-300 μm in thickness (20 μm preferred) with a large central hole of approximately 2 mm in hydraulic diameter (shown in FIG. 1). It is desirable that the third disc 3 be thin relative to the length of the metering chamber in order to minimise the dead volume between the first disc 1 and second disc 4, but not so thin (i.e. with a thickness less than the hydraulic diameter of the orifices in the nozzle) that it contributes to viscous losses.

The three elements 1, 3 and 4 are preferably made of the same class of material, i.e. a metal, in order to facilitate welding together of the elements 1, 3 and 4, preferably by laser welding. The three elements 1, 3 and 4 are preferably made of stainless steel such that they are stiff and strong and can resist pressures of approximately 250 bar without substantial plastic deformation. It should be noted, however, that stiff polymers such as polyoxymethylene (for example, delrin™) or polycarbonate could also be used, particularly if the first disc or element (nozzle plate) 1 is also well supported by an external part in use, which prevents the nozzle from bulging outwards under pressure.

In the preferred embodiment, it is desirable that the elements are laser welded together using a full seam weld in order to provide a hermetically sealed unit, such that liquid forced through the filter inlet can only exit through the nozzle outlet. However, laser spot welds could be used to affix the elements together, as long as any gaps between any two consecutive elements are smaller, in the plane substantially perpendicular to the planes of the two consecutive elements, than the diameter of the orifice 2 of the first disc 1, such that particles which are bigger than the orifice 2 of the first element 1 cannot get through and block the orifice 2. In other words, the perpendicular distance between any two consecutive elements is less than the diameter of the largest orifice 2 of the first perforate element 1. In this instance, external sealing to a metering chamber will still be required, if the nozzle assembly is to be attached to a metering valve.

The perpendicular distance between any two consecutive elements 1, 3 and 4 may always be less than the diameter of the largest orifice 2 of the first perforate element 1 or the largest orifice 5 of the second perforate element 4 across the entirety of an overlapping region of the first and second perforate elements. The perpendicular distance between the first perforate element 1 and second perforate element 4 may always be less than the largest diameter of the first perforate element 1, or the largest diameter of the second perforate element 4, across the entirety of an overlapping region of the first and second perforate elements 1 and 4.

This nozzle assembly has a low pressure drop through the filter (second perforate element 4), has a very small dead volume, and is robust to high pressure. The viscous pressure drop across the filter (second perforate element 4) is governed by the following equation:

$$P = \frac{128 μ LQ}{n π {d}^{2}}$$

where μ is the dynamic viscosity of the liquid, L is the thickness of the filter (second perforate element 4), d is hydraulic diameter of each orifice, n is the number of orifices, and Q is the flow rate through the filter (second perforate element 4). The pressure drop across a typical filter (second perforate element 4), as described above, is equal to 0.3 bar for a flow rate of 10 μL/s of water, which is orders of magnitude lower than the 80 bar required to force the fluid through the nozzle (first perforate element 1). The dead volume in between the nozzle (first perforate element 1) and the filter (second perforate element 4) is only 0.6 μL, when the intermediate element has a thickness of 50 μm and diameter of 4 mm, which is a fraction of a typical metered volume of a pump spray (10-100 μL), and hence is unlikely to have a significant impact on spray performance. Lastly, the nozzle plate is well supported and will only deflect by less than 10 μm under pressures of up to and including 100 bar.

The nozzle assembly shown in in FIG. 1 may be configured such that an impingement element lies in front of the nozzle outlet, as described in international application number PCT/GB2015/051413, such that liquid forced through the nozzle breaks up into a slow moving aerosol.

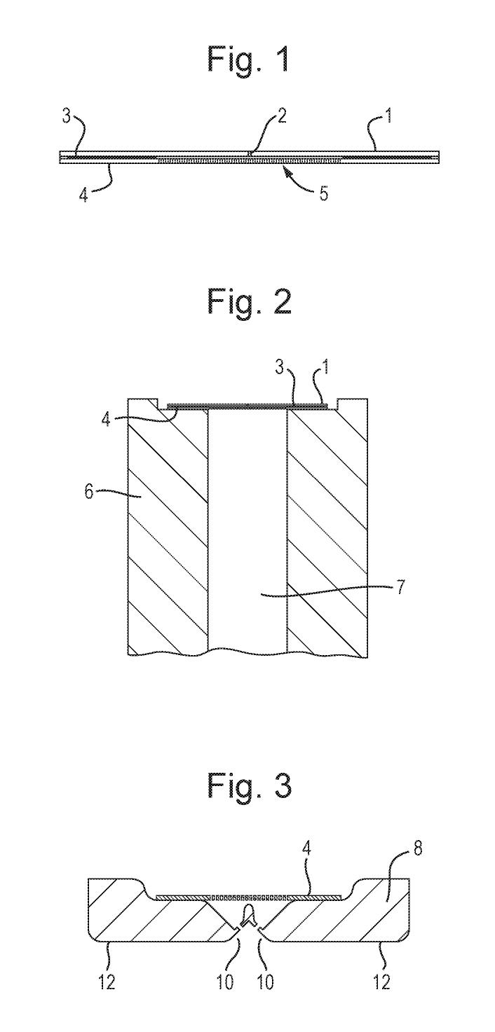

FIG. 2 shows an embodiment of the nozzle assembly where, in addition to being laser welded together, the three elements or discs 1, 3 and 4 of the nozzle assembly are laser welded to a tube 6 that defines a metering chamber 7. This is advantageous as it provides a simple method of supporting the nozzle assembly with minimal compliance and dead volume. The nozzle assembly is sealed to the metering chamber 7, with no substantial additional cost. Furthermore the nozzle assembly is supported from the inlet (filter) side, so there are no additional structures that could interfere with the emitted jet or aerosol and receive deposition of the aerosol.

FIG. 3 shows an alternate embodiment of the nozzle assembly, which does not have an intermediate supporting element. A filter element 4, similar to that described previously, is welded to a first perforate element in the form of a moulded part 8, which may be made of plastic that acts as a nozzle element. The moulded part 8 comprises one or more thin walled sections 10, typically of less than 200 μm in thickness, supported by one or more thick walled sections 12, typically of greater than 200 μm in thickness. The moulded part 8 comprises two thin walled sections 10 with outer surfaces angled at 90 degrees relative to each other, as shown in FIG. 3,

The moulded part 8 comprises at least two opposing holes, in the one or more thin walled sections 10, with hydraulic diameters of 5 to 100μm, typically 30 μm. The axes of the at least two opposing holes are at an angle of between 55 and 125 degrees, preferably 90 degrees, relative to an external surface of their respective thin walled sections 10, such that the projected areas of the holes at least partially intersect at the outlet side of the nozzle. As such, liquid forced through the opposing holes of the nozzle forms into two impinging jets, leading to break up of the liquid into a slow moving aerosol.

The embodiment shown in FIG. 3 provides a very low-cost impinging jet nozzle, with a built in filter that is easy to fabricate. The orifices in the filter can be laser drilled at the same time as the filter is being laser welded to the plastic moulded part. The nozzle assembly is substantially lower cost than other impinging jet nozzles with an integrated filter, such as those described in U.S. Pat. No. 7,896,264.

Any of the nozzle assemblies described herein could be incorporated into a nebuliser, inhaler or spray device for nasal, ophthalmic, or topical therapy.

Any of the nozzle assemblies described herein could be incorporated into a spray device comprising an impaction surface, wherein the first perforate element 1 is arranged in the spray device such that, in use, the liquid emitted from the first perforate element 1 impacts the impaction surface that is located downstream of the nozzle outlet.

The filter (second perforate element 4) described herein provides minimal flow resistance compared with that of the nozzle (first perforate element 1), i.e. the flow path through the nozzle is small to minimise viscous losses. As such the filter should be thin.

In a spray pump comprising the nozzle assembly, the filter is also located near the nozzle orifices for two reasons. Firstly, the filter must capture any wear particles that could be generated in the operation of the spray system. Secondly, it is desirable to limit any compliance and dead volume in the spray system to achieve consistent spray properties. When a constant force is applied to a typical spray pump, the force compresses any compliant seals and any trapped air bubbles at the same time as forcing liquid through the nozzle. When the force is removed from the system, the compressed seals and air continue to apply a pressure to the liquid that gradually decays with time. Liquid can continue to be forced through the nozzle for several seconds afterward, leading to undesirable continued droplet distribution.

There is a need for a low cost (<0.05 USD) and compact (e.g. containable in a millimetric envelope) valve in series with a filter and/or a nozzle (such as a sub-micrometric nozzle), with a minimised assembly dead volume. This is achieved in the present valving and filtering device by combining the multiple functions into one single lo component. Large volume manufacturing (e.g. millions of units per year) means the device must be robust to manufacturing tolerances.

The valving and filtering device consists of two perforated members attached together, one of which more flexible than the other.

The valving aspect of the valving and filtering device encompasses but is not reduced to two key types of functions: (i) check valve and (ii) flow regulation. These functions are useful independently and in combination.

The ‘check valve’ function, also known as ‘one-way valve’, sets a preferential direction to the flow. In the so called ‘forward direction’, the valve opens increasingly as the flow rate increases, thereby lowering its resistance to fluids. In the so called ‘reverse direction’, the valve closes increasingly as the pressure drop across it increases, thereby increasing its resistance to fluids.

The ‘flow regulating’ function acts on the magnitude of the flow. For example the valve can be made to close increasingly as the flow rate increases.

Controlling the size of the holes of at least one of the two perforated members enables a filter to be integrated in the valve. Controlling the hole(s) of the flexible member, e.g. their size or shape, enables one/or many nozzle(s) to be integrated in the valve. A filter-valve-nozzle can be made e.g. by creating many holes of controlled size on the less-flexing member and one or several hole(s) in the flexing member.

This valving and filtering device enables a valve and a filter and/or a nozzle to be combined into only one component. The benefits are low cost and size, minimum dead volume, and easy tuneability of many parameters of interest for the valving, filtering, and nozzling functions.

The valving and filtering device is beneficial in all cases by reducing the filter and valve to a single component. In the last example described herein, the valving and filtering device can be made even more beneficial by combining all three functions, valving, filtering and nozzling, into a single component.

Combining several parts into one has several benefits. Firstly it helps reduce the overall size of the assembly. Secondly it helps minimising the cost of manufacture by reducing the bill of materials and decreasing the assembly cost. Thirdly it enables reduction of the dead volume below what most assemblies can achieve (<1 μL).

### Valving and Filtering Device

The valving and filtering device comprises a flexing (e.g. flexible) member attached to a less-flexing member. The flexion is preferably elastic, although the flexion may also be plastic deformation. Preferably, apertures of the non-flexing member are not all aligned with apertures of the flexing member at rest. In other terms, the area of the intersection of the projected areas of the apertures of the flexing member with the apertures of the non-flexing member (projected on a surface normal to the apertures of one of the members) is strictly less than the minimum total aperture area of the individual members. However, a full alignment could still yield the desired functions, due to inlet and outlet pressure losses through the holes.

### Check Valve Function

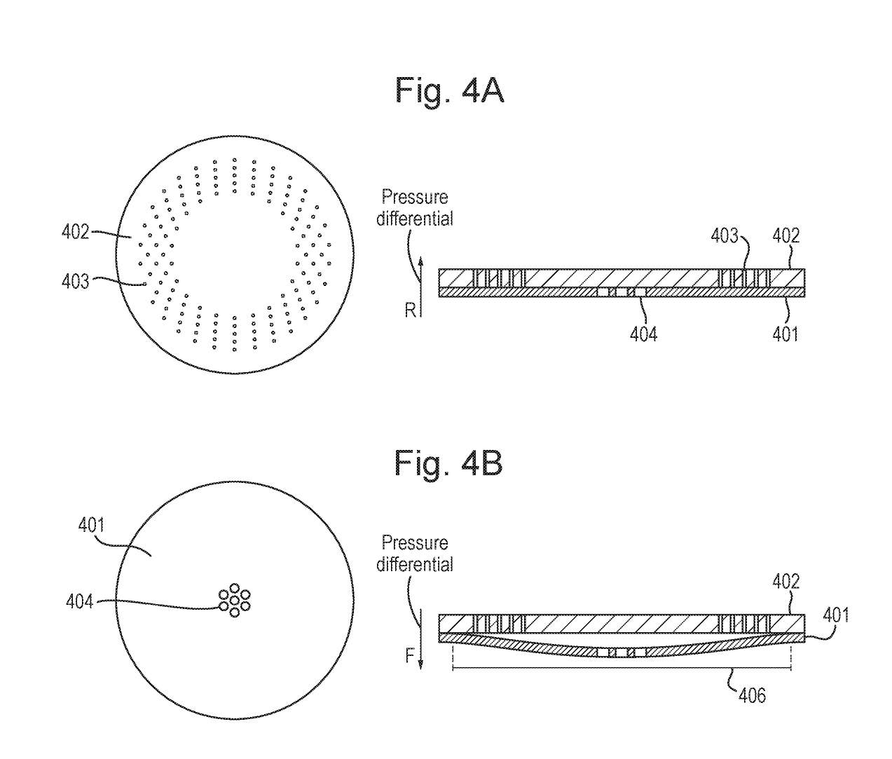

One aspect of the valving and filtering device is the provision of a ‘check valve function’. The check valve function is preferably associated with a filter. One example embodiment can be seen in FIG. 4. FIGS. 4, 4A and 4B depict a valving and filtering device comprising a flexing member 401, in the form of a first perforate element 401, and a less-flexing member 402, in the form of a second perforate element. The flexing member 401 comprises an array of apertures 404 and a less-flexing member 402 also comprises an array of apertures 403. FIG. 4 shows a top down views of the flexing member 401 and the less-flexing member 402. FIG. 4A depicts the valving and filtering device when a flow is forced in a reverse direction R, shown as arrow R in FIG. 4A. FIG. 4B depicts the valving and filtering device when a flow is forced in a forward direction F, shown as arrow F in FIG. 4B.

The apertures 403 of the less-flexing member 402 may each have a hydraulic diameter smaller than that of the smallest aperture 404 of the flexing member 401. In this way, the less-flexing member 402 acts as a filter in the same manner as the second disc or element 4 shown in FIGS. 1 to 3. Indeed, the less-flexing member 402 may have any of the above outlined features of the second disc or element 4 shown in FIGS. 1 to 3.

The perforations of both of one or more of the flexing or less-flexing members may not be fully aligned, i.e. they are partially misaligned or fully misaligned or they are partially overlapping or they do not overlap at all.

The perforations of one or more of the flexing or less-flexing members may have round or elliptical cross-sections.

Only the size of the largest perforation may be controlled, for one or more of the flexing or less-flexing members.

When a flow is forced in the forward direction F, it enters the valving and filtering device through the apertures 402 of the less-flexing member 402, which exerts a positive pressure on the flexing member 401. Above a certain pressure threshold, known to those skilled in the art as “cracking pressure”, flexing member 401 is caused to flex away from less-flexing member 402, until a flow path of a low enough resistance is opened between the apertures 403 of the less-flexing member 402 and the apertures 404 of the flexing member 401, as shown in FIG. 4B. This flow path is called the ‘valve opening’. Typically, the ‘valve opening’ opens increasingly as the flow rate increases, starting, for example, from a cracking pressure and up to a certain point.

When the flow is forced in the reverse direction R, flexing member 401 is pushed against less-flexing member 402, which will constrict/eliminate the flow path between the apertures on the two different members, as shown in FIG. 4A, thereby closing the ‘valve opening’ until a ‘closed position’ is reached, which is shown in FIG. 4. In the said ‘closed position’, the ingress and/or egress of fluids are impeded. For some applications, the constriction is total and creates a face seal, so no fluid can pass through. An example embodiment is depicted in the open position FIG. 4B and closed position FIG. 4A.

The cracking pressure would most of the time be positive, which means that the valve is sealed at rest, e.g. is in the position shown in FIG. 4A at rest, and opens under a push in the forward direction F.

The cracking pressure can alternatively be slightly negative, which means that the valve is open at rest, e.g. is in the position shown in FIG. 4B at rest, but seals above a given ‘reverse’ pressure.

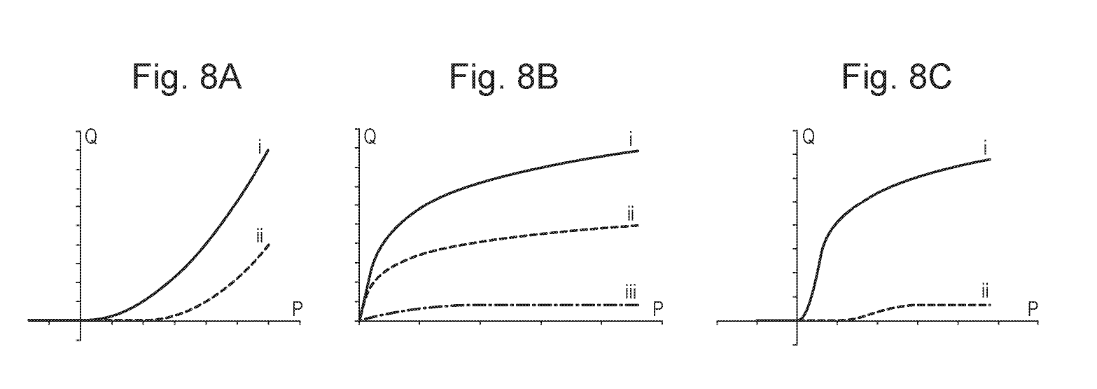

An illustration of near zero cracking pressure, line (i), and positive cracking pressure, line (ii), is shown in FIG. 8A, in a flow rate Q vs. pressure p graph.

### ‘Regulating Function’

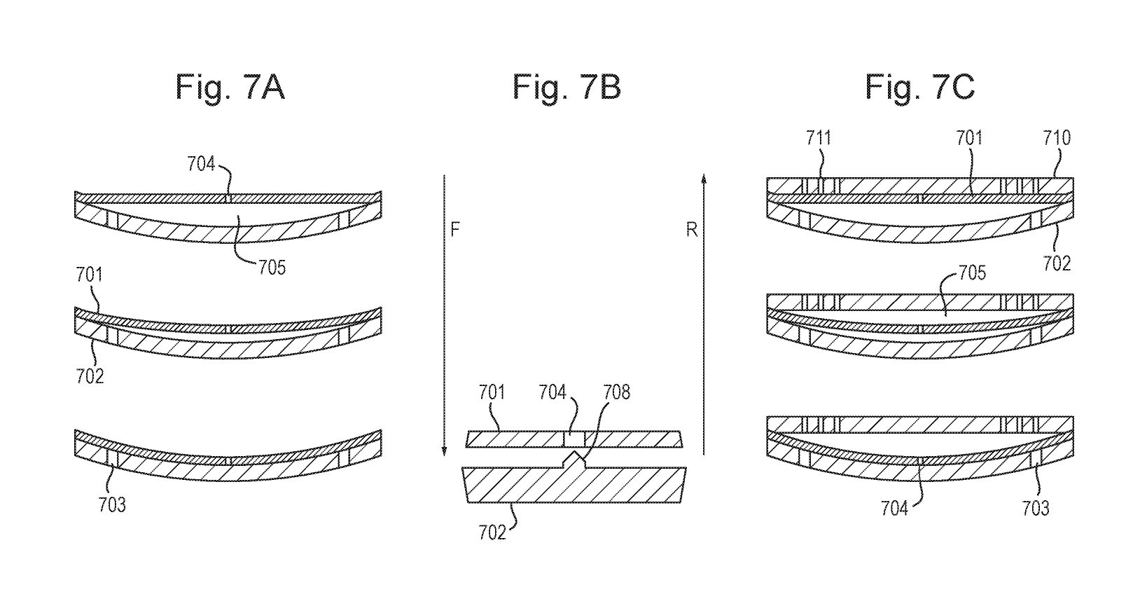

Another aspect of the valving and filtering device, shown in FIGS. 7A, 7B and 7C, involves regulating the flow rate. The regulating function may or may not be associated with a filter. For example, the flow can be increasingly restricted as the incoming flow rate increases by having a flexing member 701, comprising one or more apertures 704, closing increasingly towards a less-flexing member 702, comprising one or more apertures 703, as the incoming flow rate increases in a forward direction F.

The operation is similar to the check valve function previously described. In this kind of embodiment, illustrated in FIG. 7, the flexing member 701 is increasingly pushed towards the less-flexing member 702 as the incoming flow rate increases in the forward direction F, which increasingly restricts the flow path/valve opening 705.

In the specific embodiment shown in FIG. 7A, three depictions of the valving and filtering device are shown. The top depiction of the valving and filtering device shows the valving and filtering device at a lower incoming flow rate in the forward direction F where the ‘valve opening’ 705 is fully open. The bottom depiction of the valving and filtering device shows the valving and filtering device in at an incoming higher flow rate in the forward direction F where the ‘valve opening’ is closed. Finally, the middle depiction of the valving and filtering device shows the valving and filtering device in an intermediate position where the ‘valve opening’ is in between its open and closed positions. The incoming flow rate is higher than that of the top depiction and lower than that of the bottom depiction of the valving and filtering device.

Flow path restriction can be achieved through various means. In one embodiment, a flow path 705 is formed between two adjacent members: an upstream more flexible member 701, and a downstream less flexible member 702. The flow path is between at least one aperture 704 in the flexible member 701 and at least one aperture 703 in the less flexible member 702. As the flow rate increases, the more flexible member elastically deforms to fully or partially fill this flow path, increasing flow resistance and limiting flow rate.

This embodiment is illustrated in FIG. 7A; three depictions of the valving and filtering device are shown. The top depiction of the valving and filtering device shows the valving and filtering device at a lower incoming flow rate in the forward direction F where the ‘valve opening’ 705 is fully open. The bottom depiction of the valving and filtering device shows the valving and filtering device in at an incoming higher flow rate in the forward direction F where the ‘valve opening’ is closed. Finally, the middle depiction of the valving and filtering device shows the valving and filtering device in an intermediate position where the ‘valve opening’ is in between its open and closed positions. The incoming flow rate is higher than that of the top depiction and lower than that of the bottom depiction of the valving and filtering device.

An advantage of this flow regulator is that, via the material selection and geometry of the parts, the resistance versus flow rate profile can be tuned to match the exact requirements desired. Both the material choice and the thickness of the members can be used to modify their flexural stiffness—which dictates the relationship between the applied flow rate and the level of deformation experienced by each member. Similarly, the spacing of apertures 703 and 704 and the initial height and length of the flow path 705 are design variables which can be used to customise flow regulator behaviour.

Rather than constricting an entire flow path, localised constrictions can also be used to produce a flow governing affect and provide a similar level of customisability through geometry, for instance: protruding features 708 could be provided on the less-flexible member adjacent to some of all of the apertures 704 on the flexible member (or vice versa, or protrusions and holes could be interdigitating, i.e. protrusions on both the flexible member and the less-flexible member, just in front of corresponding holes on the other member). At larger flow rates, the apertures flex onto the corresponding protrusion, becoming increasingly blocked and limiting the flow rate. This is illustrated in FIG. 7B.

In a similar embodiment, instead of protrusions directly blocking the apertures, one set of features could be provided on the downstream face of the more flexible member and another set of interlocking features on the upstream face of the less flexible member. At increased flow rates, the features on each face increasingly interlock, providing a tortuous path and limiting flow. In these embodiments, the positions and dimensions of these interlocking features, as well as material parameters, can be used to customise regulator flow response.

The valving and filtering device could be designed such that it limits flow to zero (i.e. provide a full seal at a specified flow rate) or has a minimum flow—for instance if a small bypass channel was set up either in parallel with the flow regulator, or within the surface of a member 701/702 itself, limiting the minimum size of the flow channel 705.

Further, the flow regime could be used to control the resistance profile with flow rate: in laminar flows the relationship between flow rate and pressure drop is linear, whereas in turbulent flows the same relationship is quadratic. Specific features, like the hydraulic diameter of the inlet aperture(s) 704 or the surface roughness of both members 701, 702 could be used to promote a particular regime.

Examples of different flow use are given on FIG. 8B. Profile (iii) shows a bounded flow rate—where there is a point beyond which increases in applied pressure do not result in an increased flow rate. Profiles (i) and (ii) are two examples of restricted but unbounded flow rates. In these examples, increasing the applied pressure results in a flow rate increase, but this increase the rate of increase diminishes beyond a certain flow rate. Said in other terms, the flow resistance of the regulator increases with flow rate.

### Combinations of Check Valves and Flow Regulators

Various combinations of the one-way filter-valve type and the flow regulating valve can be assembled together, e.g. using three members 710, 701 and 702 in a stiff-flexible-stiff stack fashion, as illustrated in FIG. 7C. A first less-flexible member 710, further comprising apertures 711, is attached to a flexible member 701, further comprising apertures 704, which is in turn attached to a second less-flexible member 702, further comprising apertures 703. The arrangement is the same as the arrangement shown in FIG. 7a, with the addition of a further less-flexible member 710.

The apertures 703 of the less-flexing member 702 and the apertures 711 of the less-flexing member 710 may each have a hydraulic diameter smaller than that of the smallest aperture 704 of the flexing member 701. In this way, the less-flexing members 702 and 710 may act as a filter in the same manner as the second disc or element 4 shown in FIGS. 1 to 3. Indeed, the less-flexing members 702 and 710 may have any of the above outlined features of the second disc or element 4 shown in FIGS. 1 to 3.

The perforations of both of one or more of the flexing or less-flexing members may not be fully aligned, i.e. they are partially misaligned or fully misaligned or they are partially overlapping or they do not overlap at all.

The perforations of one or more of the flexing or less-flexing members may have round or elliptical cross-sections.

Only the size of the largest perforation may be controlled, for one or more of the flexing or less-flexing members.

In the specific embodiment shown in FIG. 7C, three depictions of the valving and filtering device are shown. The top depiction of the valving and filtering device shows the valving and filtering device at a lower incoming flow rate in the forward direction F (i.e. a pressure of less that P1 is applied across the device, where P1 is the cracking pressure) where the ‘valve opening’ 705 is in a closed position as the flexible member 701 is abutting the first less-flexible member 710.

The middle depiction of the valving and filtering device shows the valving and filtering device in an intermediate position where the ‘valve opening’ is in between its open and closed positions. The incoming flow rate is higher than that of the top depiction (i.e. a pressure of more than P1, but less than P2, is applied across the device) where the ‘valve opening’ 705 is in an open position.

The bottom depiction of the valving and filtering device shows the valving and filtering device at an incoming flow rate which is applying a pressure of greater than P2 (P2 is greater than P1) across the device in the forward direction F where the ‘valve opening’ is closed.

In the specific embodiment shown in FIG. 7C, applying a pressure across the system in the forward direction F pushes the flexing member 701 away from the first less-flexible member 710 and toward the second less-flexible member 702. Above a pressure P1, the cracking pressure, it opens the valve, as shown in the middle depiction of the valving and filtering device in FIG. 7C. Above a pressure P2 (P2>P1), the flow path starts to be restricted between the flexible member 701 and the second less-flexible member 702. This increases the airflow resistance, and restricts the flow. As the pressure increases, eventually the valve opening is closed, as shown in the bottom depiction of the valving and filtering device in FIG. 7C. This example arrangement enables a preferential direction for the flow to be set, as well as enabling the high flow rates to be controlled, in combination with a valving function. This is illustrated on FIG. 8C. Both lines show a combination of both the check valve function and the regulation function, i.e. blocking the fluid in the reverse direction R while letting the fluid flow in the forward direction F above a chosen cracking pressure, and regulating the high flow rates. Line (i) has a near zero cracking pressure and restricts, but does not fully prevent, the increase of flow rate with pressure. Line (ii) has a strictly positive cracking pressure, and its flow rate is maintained below a set maximum bound.

### Tuneability—Manufacturing Considerations

The valving and filtering device is very well suited to mass manufacturing, as both members could be, for example, moulded and welded together, leading to a cost of a few $c per unit.

In addition, the valving, filtering and nozzling functions of the valving and filtering device can be tuned easily by varying one or a few dimensions.

The operating parameters include, but are not limited to, filtering size, the relation between pressure and flow rate in both the forward and reverse direction (includes the cracking pressure and the leak rate; relationship also called flow resistance), the minimum and maximum working pressures in both directions (e.g. related to straight rupture or fatigue failure), and in presence of nozzles, jet properties (such as jet diameter and stability).

The levers to tune the operating parameters include, but are not limited to, the apertures numbers, sizes, 3D shape and positions, the material thickness of each member, the diameter at which the two members are attached together, the material mechanical properties, etc.

The methods to pull the levers to tune the operating parameters include, but are not limited to:
using an easy to tune perforation means, such as a laser driller to modify the apertures numbers, sizes and positions;
using means to attach the two members together for which changing the diameter of attach is convenient—this could involve for example using welding tools of various diameters grouped on an automatic rotary stage; and
swapping a sheet of a given material, thickness and diameter for another.

These methods can be used separately or in combination. Below are more details.

The filtering cut-off is directly linked to the hole size of one or both perforate members. The nozzle functions, e.g. creation of a jet, are also defined by the hole size and shape. The resistance and cracking pressure of the valving function can be controlled by many tuneable parameters linked to flexibility and force generation, as described below.

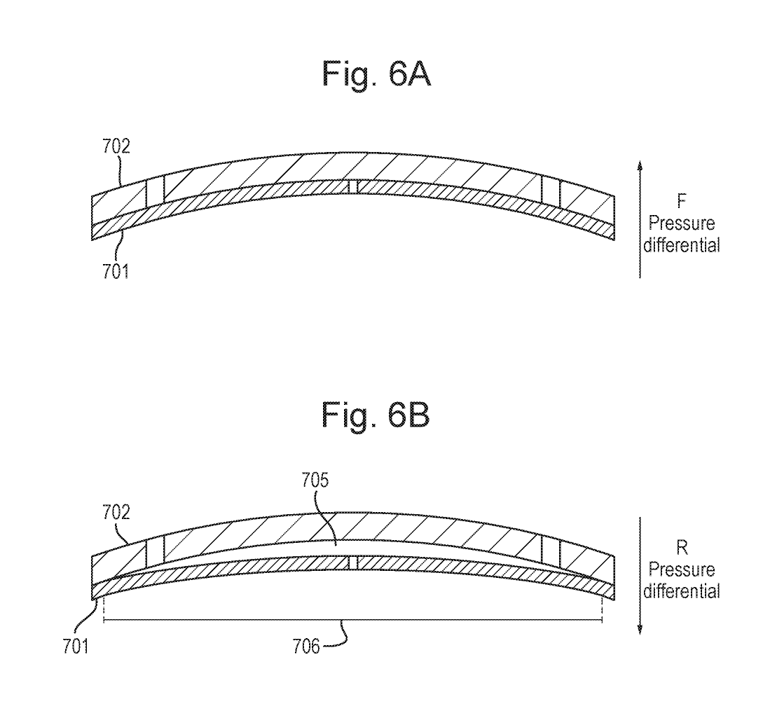

The relative flexibility of the flexible members 401, 701 and the less-flexible members 402, 702, 710 of the valving and filtering device described herein is influenced by the relative thicknesses of the members, the relative material properties of the members (e.g. Young elastic modulus and Poisson's ratio), the diameter at which the members are mechanically bonded together 406, 706 (as shown in FIGS. 4 and 6), and the existence of any pre-stress/shape of one or both members at rest, as illustrated in FIG. 6.

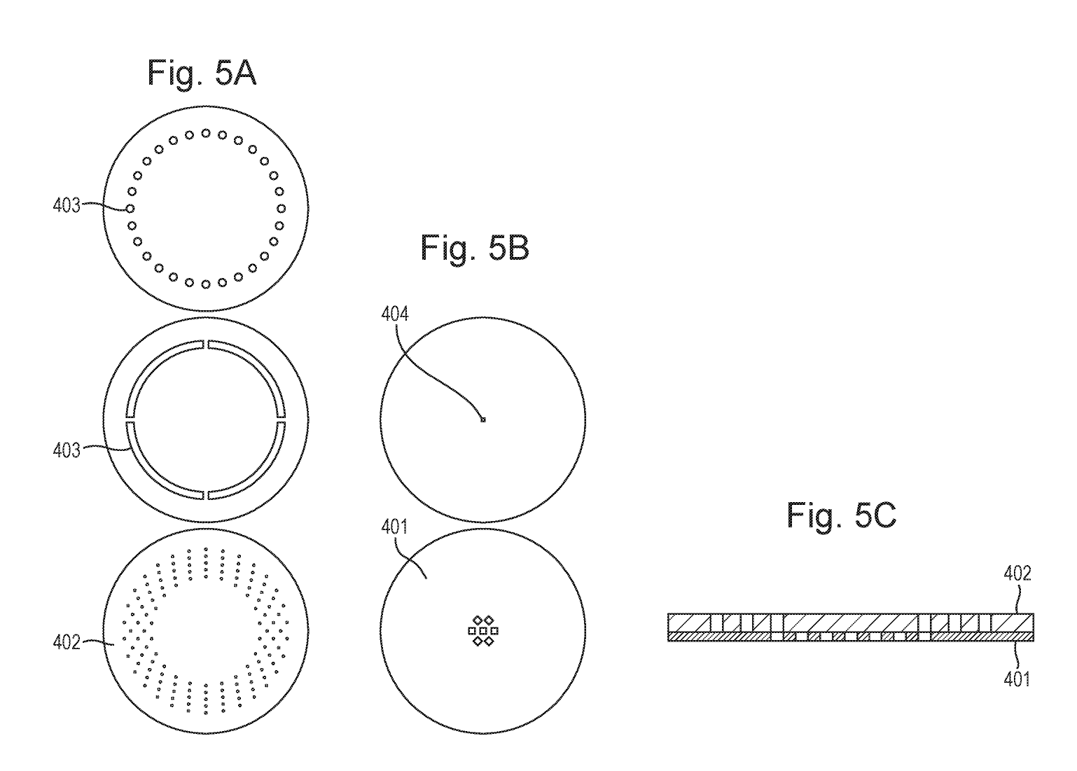

On the force generation side, one of several levers, is the aperture/hole areas and relative positions 403, 703 and 404, 704 on each member, examples of which are depicted on FIG. 5. FIG. 5A shows examples of the less-flexible member 402, shown also in FIG. 4, with various hole numbers, positions and shapes. FIG. 5B shows examples of the flexible member 401. FIG. 5C shows a section of both members 401 and 402 at resting. The 3D profile of each of the perforations also affects the resulting force, as it affects the pressure drop generated by a viscous fluid flowing through. For example, a conic hole will have a different pressure drop in the forward flow direction F than in the reverse flow direction R.

The preferred way in which the valving and filtering device offers an exceptionally quick, cheap and flexible customisation is the control of aperture number, size, 3D shape and relative position, which can be achieved without any modification to the production and assembly lines with several perforating processes. Taking laser drilling as an example of perforating process, hole positions and numbers can be defined by moving the focal point of the laser in a plane normal to the perforation direction. Each hole size and 3D shape can be tuned by controlling the number and power of each pulse, and whether the laser beam is focusing inside the hole, upstream or downstream of it. This type of features can be electronically controlled. If desirable, it can be directly linked to online orders, and enables variations on each unit to be managed. Compared to methods found in the industry, this enables:
(i) storing of parts to be avoided, such as would be necessary with a spring-ball check valve system;
(ii) operation over a continuum rather than with discreet parameters, such as is necessary if components have to be switched or if a certain drill tool is used;
(iii) a natural implementation on the assembly line enabling ‘on the fly tuneability’, while switching components would require substantial effort to enable swapping of valves on the fly or would require a production interruption; and
(iv) keeping of the same assembly lines, while other designs may require different machines to assemble different types of designs.

Other parameters can be tuned by using a more common approach, but that still offers the advantages (i) and (iv). An example is the control of the diameter at which the two members are attached together, which affects the geometric stiffness of the part. This affects many parameters such as the cracking pressure or the pressure range. There are many ways in which this could be done, for instance by having tools (e.g. welding) of various sizes on an automatic tool changer (e.g. on a rotary stage).

Swapping plates of varying thickness and material properties of one or several members is yet another type of tuneable parameter, as it would affect the cracking pressure and the maximum operating pressures. Although this last type of customisation does not have all the advantages of the previous method, it keeps the advantage (iv) of keeping the same assembly line and simply requires modification of parameters of the production line (e.g. time of ultrasonic welding if it is used to attach the two members together) which can be changed in a quick and automatic fashion.

Overall, the valving and filtering device enables complete tuneability of operating parameters, and all these parameters can be customised with all the advantages (i), (ii), (iii) and (iv).

### Environments

This valving and filtering device enables the claimed functions to be performed using any sort of fluid, whether it is liquid, gaseous or other (e.g. supercritical).

The range of pressure over which the valving and filtering device works and is useful is very wide. Typically the pressures encountered in practice range from a few Pascals to hundreds of bars, but this does not constitute a limitation to the valving and filtering device. For the ‘check valve’ function, the maximum pressure in the reverse direction is likely to be set by the rupture or yield properties of the material in the zones of highest stress concentration (e.g. where the flexing member is attached). In the forward direction the maximum pressure is linked to the resistance of the valve in the forward direction and, therefore, the pressure drop it generates at a certain flow rate.

If sealing properties at low pressure are key, unwanted plastic deformation should be avoided, or more precisely pressures causing yield should be designed for.

1. A nozzle assembly, configured to receive fluid at pressures of greater than 1 bar, the nozzle assembly comprising:
a first perforate element comprising one or more orifices, each orifice having an inlet and an outlet and a diameter of no more than 100 μm; and
at least one second perforate element further comprising a plurality of orifices of a smaller size than the one or more orifices of the first perforate element and having a larger number of orifices than the first perforate element;
wherein the second perforate element is configured to act as a filter,
wherein the second perforate element is attached to the first perforate element,
wherein a perpendicular distance between the first and second perforate elements is less than the diameter of the largest orifice of the first perforate element.

2. A nozzle assembly through which fluid is forced at pressures greater than 1 bar, the nozzle assembly comprising:
a first perforate element comprising one or more orifices, each orifice having an inlet and an outlet and a diameter of no more than 100 μm;
at least one second perforate element further comprising a plurality of orifices of a smaller size than the one or more orifices of the first perforate element, the second perforate element having a larger number of orifices than the first perforate element, the second perforate element being arranged to act as a filter; and
at least one intermediate element that separates the first and second perforate elements,
wherein the second perforate element is attached to either the intermediate element or the first perforate element,
wherein a perpendicular distance between one or more of any two consecutive perforate and/or intermediate elements is less than the diameter of the largest orifice of the first perforate element.

3. A nozzle assembly according to claim 2, wherein the perpendicular distance between the first and second perforate elements is such that there is no route for a particle of larger size than the one or more orifices of the first perforate element to enter between the first and second perforate elements.

4. A nozzle assembly according to claim 2, wherein the perpendicular distance between the first perforate element and the at least one intermediate element and/or the perpendicular distance between the second perforate element and the at least one intermediate element is such that there is no possible route for a particle of larger size than the one or more orifices of the first perforate element to enter between the first perforate element and the at least one intermediate element and/or between the second perforate element and the at least one intermediate element.

5. A nozzle assembly according to claim 2, wherein the perpendicular distance between the first and second perforate elements is always less than the largest diameter of the first or the second perforate element across the entirety of an overlapping region of the first and second perforate elements.

6. A nozzle assembly according to claim 2, wherein the first and second perforate elements are hermetically sealed together such that, in use, fluid can only flow between the orifices of the second perforate element and the one or more orifices of the first perforate element.

7. A nozzle assembly according to claim 2, wherein the first perforate element comprises at least two orifices, wherein [[the]] projected areas of the at least two opposing orifices at least partially intersect at an outlet side of the first perforate element, wherein, in use, an aerosol is generated from at least two impinging jets formed when liquid is forced through the nozzle assembly.

8. A nozzle assembly according to claim 2, wherein the second perforate element is not welded to the first perforate element.

9. A nozzle assembly according to claim 2, wherein the second perforate element is welded to the first perforate element.

10. A nozzle assembly according to claim 2, wherein one or more of the elements is attached to another of the elements, using one or more of bonding, clamping, gluing, soldering, brazing, co-moulding, welding, laser welding, ultrasonic welding, electron beam or thermal welding process.

11. A nozzle assembly according to claim 2, wherein the at least one second perforate element is disposed on a liquid side of the nozzle assembly.

12. A spray device comprising a nozzle assembly according to claim 2, the spray device further comprising an impaction surface, wherein the first perforate element is arranged in the spray device such that, in use, the liquid emitted from the first perforate element impacts the impaction surface that is located downstream of the nozzle assembly.

13. A metering chamber for a spray device comprising:
the nozzle assembly of claim 2; and
a tube defining a cavity,
wherein the nozzle assembly is attached to the tube with the second perforate element facing toward the cavity defined by the tube.

14. A nebuliser or inhaler comprising the nozzle assembly of claim 2.

15. A device for nasal, ophthalmic, or topical therapy comprising the nozzle assembly of claim 2.

16. A valving and filtering device comprising
a first perforate member; and
a second perforate member attached to the first perforate member,
wherein the first perforate member is more flexible than the second perforate member and,
wherein orifices of at least one of the first and second perforate members have a hydraulic diameter between 0.1 μm and 1 mm per hole.

17. A valving and filtering device according to claim 16, wherein the orifices of both of the first and second perforate members have round or elliptical cross-sections.

18. A valving and filtering device according to claim 16, wherein only the size of the largest orifice is controlled, for either or both the first and second perforate members.

19. A valving and filtering device according to claim 16, wherein the first perforate member is arranged to abut the second perforate member in such a way that any flow of fluid between the orifices of first perforate member and the orifices of the second perforate member is prevented when the device is exposed to a flow of fluid in a first direction such that the device acts as a one-way valve.

20. A valving and filtering device according to claim 19, wherein the first perforate member is arranged such that when the device is exposed to a flow of fluid in a second direction opposite the first direction, the first perforate member moves away from the second perforate member, thereby allowing a flow of fluid between the orifices of first perforate member and the orifices of the second perforate member.

21. A valving and filtering device according to claim 20, the first perforate member is arranged to abut the second perforate member in such a way that any flow of fluid between the orifices of first perforate member and the orifices of the second perforate member is prevented when the device is at rest.

22. A valving and filtering device according to claim 16, wherein at least one of the first and second perforate members are perforated only with apertures of a controlled maximum size, preferably between 0.1 μm and 200 μm, and more preferably between 0.1 μm and 10 μm.

23. A valving and filtering device according to claim 16, wherein the first perforate member is configured to be pushed toward the second perforate member as flow rate increases, thereby increasingly restricting the flow.

24. A valving and filtering device according to claim 16, wherein the first perforate member comprises one or more integrated nozzle(s) of controlled size, preferably of between 0.5 μm and 1 mm and, more preferably of between 5 μm and 200 μm and, wherein apertures of the second perforate member are of a controlled maximum size and are configured to perform a filtering function.

25. A valving and filtering device according to claim 16, wherein each orifice of the second member has a smaller area and/or size than the smallest orifice on the first member.

26. A valving and filtering device according to claim 16, wherein the first perforate member is configured to be pushed away from the second perforate member as flow rate decreases, thereby decreasingly restricting the flow.

27. A valving and filtering device according to claim 22, further comprising a third perforate member,
wherein one or more of the first perforate member and the second perforate member are attached to the third perforate member,
wherein the first perforate member is more flexible than the third perforate member and,
wherein the first perforate member is disposed between the second and third perforate members.

28. The valving and filtering device of claim 27, wherein the orifices of at least one of the first, second and third perforate members are of controlled size, preferably between 0.1 μm and 5 mm.

29. A valving and filtering device according to claim 27, in which the orifices of two or more of members are not fully aligned.

30. A valving and filtering device according to claim 27, in which only the maximum orifice size is controlled, for at least one of the first, second and third perforate members.

31. A valving and filtering device according to claims 27 in which two or more of the members are attached together by a welding process.

32. A valving and filtering device according to claim 16 in which at least some orifices are laser drilled.

33. A valving and filtering device according to claim 16 in which some orifices are tapered from the inlet to the outlet.

34. A valving and filtering device according to claim 16 in which some orifices have a smaller cross-section on one side of their respective member than on the other side.

35. A valving and filtering device according to claim 16, wherein a laser driller or a laser welder are used to modify one or more valving properties of the device, which include, but are not limited to, filtering size, cracking pressure, min and max operating pressures, regulation flow rate.

36. A valving and filtering device according to claim 27, wherein at least one of the members comprises steel.

37. A valving and filtering device according to claim 27, wherein at least one of the members comprises a plastic material.

38. A valving and filtering device according to claim 27, wherein at least one of the members comprises a Mylar film or a polyethylene terephthalate film.

39. A pump, aerosol generator, jet generator, or fluid transport device comprising the valving and filtering device of claim 16.
